(19) United States
(12) Patent Application Publication
(10) Pub. No.: US 2020/0323757 A1
(43) Pub. Date: Oct. 15, 2020

(54) KIT FOR PRODUCING A BODY CARE COMPOSITION

(71) Applicant: Henkel AG & Co. KGaA, Duesseldorf (DE)

(72) Inventors: THOMAS SCHROEDER, Hamburg (DE); SOEREN SCHEELE, Pinneberg (DE); MANUELA METTE, Kleinfeld (DE)

(73) Assignee: Henkel AG & Co. KGaA, Duesseldorf (DE)

(21) Appl. No.: 16/765,425

(22) PCT Filed: Nov. 12, 2018
(86) PCT No.: PCT/EP2018/080869

§ 371 Date: May 19, 2020

(30) Foreign Application Priority Data
Dec. 14, 2017 (DE) 10 2017 222 793.1

(51) Int. Cl. A61K 8/41; A61K 8/42; A61K 8/46; A61Q 5/02

(52) U.S. Cl. CPC A61K 8/416; A61K 8/42; A61Q 5/12; A61Q 5/02; A61K 8/463

(57) ABSTRACT

A kit comprising at least two base liquids for the preparation of a body treatment composition is provided. In an exemplary kit, each base liquid contains at least about 10% by weight of a surfactant mixture comprising anionic surfactants, amphoteric surfactants and nonionic surfactants. In exemplary embodiments, the surfactant mixture comprises alkyl ether sulfates, alkyl amidoalkyl betaines and alkamide monoethanolamines.

### CROSS-REFERENCE TO RELATED APPLICATION

This application is a U.S. National-Stage entry under 35 U.S.C. § 371 based on International Application No. PCT/EP2018/080869, filed Nov. 12, 2018, which was published under PCT Article 21(2) and which claims priority to German Application No. 10 2017 222 793.1, filed Dec. 14, 2017, which are all hereby incorporated in their entirety by reference.

### TECHNICAL FIELD

The present disclosure concerns a kit for preparing a body treatment agent, in particular a hair treatment agent, and a mixing device for producing a liquid mixture, comprising such a kit.

### BACKGROUND

In many areas of daily life there is a continuing trend towards personalized programs that respond to the individual requirements and needs of a customer or user. This is the case, for example, in the areas of nutrition or health care and also in the field of personalized cosmetics. This enables the consumer or user of cosmetic treatment products to receive individual treatment instructions and cosmetic treatment products that are specifically tailored to the individual needs of, for example, his hair or skin and thus enables a particularly high degree of effectiveness.

A hairdresser may determine the current hair condition (damaged, dull, brittle, etc.) of a customer with the help of his experience and/or with suitable analysis equipment. Usually, he then resorts to products available in the hairdressing salon when choosing the appropriate hair treatment product.

Conventional, commercially available hair treatment products are available as a limited product range for a given application. For example, each manufacturer of shampoos, deep conditioners or hair colors provides a limited range of products. Even though a user's hair condition may be known, it may be impossible to achieve an optimal treatment result, but usually only a result close to the desired result can be achieved.

It is therefore desirable to produce a cosmetic hair treatment product for the user on site, for example in a hairdressing salon or at a point of sale, on the basis of the determined hair condition and individually tailored to the hair condition.

However, manually mixing different hair treatment products, for example, components for a shampoo, conditioner, deep conditioner or hair dye, is a challenge for the hairdresser or the user himself. An optimal mixing result may only be achieved if the individual components are mixed exactly in their predefined proportions. For example, when manually mixing several components from bottles, a portion of one component may remain in the bottle excessively, so that the mixture may lack a portion of one component.

By employing a mixing device comprising a plurality of viscous liquid reservoirs containing body treatment product ingredients, a plurality of pumps for conveying the viscous liquids and a static mixer, it is possible to produce a desired body treatment product accurately and easily by mixing at least two liquids.

There are numerous requirements to be met by the liquids in the storage tanks: for example, they must be easy to mix, pump well and precisely and he stable in storage. In addition, they should be able to contain high concentrations of active ingredients and/or additives, such as oils, perfumes, so that even small quantities of the desired hair treatment product may be prepared, for example, for a single application.

Consequently, there is a demand for a kit comprising a plurality of liquids containing ingredients of body treatment products which, when used in a mixing device, enables an individual body treatment product, in particular a hair treatment product, to be prepared for a user in a standardized and objective manner.

This task is solved by a kit comprising at least two base liquids for the preparation of a body treatment product, each base liquid containing at least about 10% by weight of a surfactant mixture comprising or consisting of anionic surfactants, amphoteric surfactants and non-ionic surfactants.

It is particularly preferred that each base liquid contains at least about 10% by weight of a surfactant mixture comprising or consisting of alkyl ether sulfates, alkyl amido alkyl betaines and alkamide monoethanolamines.

### BRIEF SUMMARY

In certain embodiments, a kit comprising at least two base liquids for the preparation of a body treatment composition is provided. In an exemplary kit, each base liquid contains at least about 10% by weight of a surfactant mixture comprising anionic surfactants, amphoteric surfactants and nonionic surfactants. In exemplary embodiments, the surfactant mixture comprises alkyl ether sulfates, alkyl amidoalkyl betaines and alkamide monoethanolamines.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has been discovered that base liquids with this surfactant mixture are very readily miscible with one another. In addition, various ingredients of body treatment products are compatible with these surfactants and may be introduced stably into the base liquids.

Preferred alkyl ether sulfates are selected from the group consisting of C₄ to C₂₄, preferably C₆ to C₁₈, particularly preferably C₈ to C₁₄ alkyl ether sulfates. The alkyl radicals may be linear or branched, linear alkyl ether sulfates being preferred.

The addition of an alkyl or alkenyl group with the suffix “eth” generally describes the addition of one or more ethylene oxide units. The number of ethylene oxide units is characterized by the addition of an integer.

Particularly preferred alkyl ether sulfates have a C₁₂ alkyl group (lauryl group) and include ammonium laureth sulfates, ammonium laureth-5 sulfates, ammonium laureth-7 sulfates, ammonium laureth-9 sulfates, ammonium laureth-12 sulfates, DEA-laureth sulfates, magnesium laureth sulfates, magnesium laureth-5 sulfates, magnesium laureth-8 sulfates, magnesium laureth-16 sulfates, MEA laureth sulfates, MIPA laureth sulfates, potassium laureth sulfates, sodium laureth sulfates, sodium laureth-5 sulfates, sodium laureth-7 sulfates, sodium laureth-8 sulfates, sodium laureth-12 sulfates, sodium laureth-40 sulfates, TEA laureth sulfates and mixtures thereof. A particularly preferred alkyl ether sulfate is sodium laureth sulfate (sodium lauryl ether sulfate).

Preferred alkyl amido alkyl betaines comprise in particular C₄ to C₂₄, preferably C₆ to C₁₈, particularly preferably C₈ to C₁₄ alkyl amido propyl betaines. The alkyl radicals may be linear or branched, linear alkyl amido propyl betaines being preferred. Alkyl amido propyl betaines which are particularly preferably contained are selected from the group consisting of capryl/capramidopropyl betaines, cocamidopropyl betaines, coco/oleamidopropyl betaines, isostearamidopropyl betaines, lauramidopropyl betaines, oleamidopropyl betaines, palmitamidopropyl betaines, ricinoleamidopropyl betaines, stearamidopropyl betaines, tallowamidopropyl betaines, undecylenamidopropyl betaines, sunfloweramidopropyl betaines and mixtures thereof, cocamidopropyl betaines (=cocamidopropyl betaine) being preferred.

Preferred alkamide monoethanolamines are selected from the group consisting of cocamide monoethanolamines (=cocamide MEA), oliveamide monoethanolamines, palm kernelamides monoethanolamines, palmamide monoethanolamines, peanutamide monoethanolamines, tallowamide monoethanolamines, wherein cocamide monoethanolamines (N-(hydroxyethyl)-coconut fatty acid amide) are particularly preferred.

It is preferred that the amount of alkyl ether sulfates is from about 7 to about 15% by weight, the amount of alkyl amidoalkyl betaines is from about 1.5 to about 4% by weight and the amount of alkamide monoethanolamines is from about 1 to about 2% by weight, in each case based on the total amount of base liquid.

It is also preferred that the surfactant mixture contains sodium lauryl ether sulfate (INCI: Sodium Laureth Sulfate), cocamidopropyl betaine (INCI: Cocamidopropyl Betaine) and N-(hydroxyethyl) coconut fatty acid amide (INCI: Cocamide Monoethanolamine).

Accordingly, it is particularly preferred that the surfactant mixture contains from about 7 to about 15% by weight of sodium lauryl ether sulfate, from about 1.5 to about 4% by weight of cocamidopropyl betaine and from about 1 to about 2% by weight of N-(hydroxyethyl)coconut fatty acid amide, in each case based on the total amount of base liquid.

Base liquids containing the triple combination of selected surfactants are structurally viscous and thus very easy to pump. The base liquids may also be dosed well and precisely. This makes it possible to prepare individual body treatment products in commercially available quantities ranging from a few ml for single use to several hundred ml, especially about 250 ml or about 400 ml.

In various forms, at least one base liquid also contains a substance selected from dialkyl sulfosuccinates and dialkyl methyl sulfosuccinates.

Preferably dialkyl sulfosuccinates and/or dialkyl methyl sulfosuccinates contained in the at least one base liquid are selected from the group consisting of dialkyl sulfosuccinates and/or dialkyl methyl sulfosuccinates in which the alkyl radicals each have 4 to 24, preferably 6 to 18, particularly preferably 6 to 14 carbon atoms. The alkyl radicals may be linear or branched, branched ones being preferred. Different or identical alkyl radicals in a molecule may be dialkyl sulfosuccinate and/or dialkyl methyl sulfosuccinate where identical ones are preferred.

Alkali metal cations, alkaline earth cations or ammonium ions, especially sodium, may preferably be used as counter ions to the sulfonic acid group. In particular, dialkyl sulfosuccinates are contained in the base liquids. The preferred dialkyl sulfosuccinates are selected from the group consisting of diethylhexyl sodium sulfosuccinates, dinonyl sodium sulfosuccinates and di-isononyl sodium sulfosuccinates, dioctyl sodium sulfosuccinates, diheptyl sodium sulfosuccinates, dihexyl sodium sulfosuccinates, dineopentyl sodium sulfosuccinates, diisoamyl sodium sulfosuccinates, dipentyl sodium sulfosuccinates, diamyl sodium sulfosuccinates, dibutyl sodium sulfosuccinates, diisobutyl sodium sulfosuccinates, dicapryl sodium sulfosuccinates, didecyl sodium sulfosuccinates, diundecyl sodium sulfosuccinates, dilauryl sodium sulfosuccinates, dicocoyl sodium sulfosuccinates, ditridecyl sodium sulfosuccinates, dipropylheptyl sodium sulfosuccinates, dicyclohexyl sodium sulfosuccinates, ammonium diethylhexyl sulfosuccinates, ammonium dinonyl sulfosuccinates, ammonium disononyl sulfosuccinates, ammonium dioctyl sodium sulfosuccinates, ammonium diheptyl sulfosuccinates, ammonium dihexyl sulfosuccinates, ammonium dineopentyl sulfosuccinates, ammonium diisoamyl sulfosuccinates, ammonium dipentyl sulfosuccinates, ammonium diamyl sulfosuccinates, ammonium dibutyl sulfosuccinates, ammonium diisobutyl sulfosuccinates, ammonium dicapryl sulfosuccinates, ammonium didecyl sulfosuccinates, ammonium diundecyl sulfosuccinates, ammonium dilauryl sulfosuccinates, ammonium dicocoyl sulfosuccinates ammonium ditridecyl sulfosuccinates, ammonium dipropylheptyl sulfosuccinates, ammonium dicyclohexyl sulfosuccinates, diethylhexyl potassium sulfosuccinates, dinonyl potassium sulfosuccinates, diisononyl potassium sulfosuccinates, dioctyl potassium sulfosuccinates, diheptyl potassium sulfosuccinates, dihexyl potassium sulfosuccinates, dineopentyl potassium sulfosuccinates, diisoamyl potassium sulfosuccinates, dipentyl potassium sulfosuccinates, diamyl potassium sulfosuccinate, dibutyl potassium sulfosuccinate, diisobutyl potassium sulfosuccinate, dicapryl potassium sulfosuccinate, didecyl potassium sulfosuccinate, diundecyl potassium sulfosuccinate, dilauryl potassium sulfosuccinate, dicocoyl potassium sulfosuccinates, ditridecyl potassium sulfosuccinates, dipropylheptyl potassium sulfosuccinates, dicyclohexyl potassium sulfosuccinates, with diethylhexyl sodium sulfosuccinates being particularly preferred.

It is preferred that the at least one base liquid also contains diethylhexyl sodium sulfosuccinate (sodium dioctyl sulfosuccinate), which is available from Evonik under the name Tego® Sulfosuccinate DO 75.

The amount of dialkyl sulfosuccinate, in particular of diethylhexyl sodium sulfosuccinate, is preferably from about 2 to about 20% by weight, more preferably from about 7 to about 15% by weight, each based on the total amount of base liquid.

In various forms, at least one base liquid may further comprise a substance with the INCI designation “Guar Hydroxypropyltrimonium Chloride”, which has a nitrogen content of from about 1.9 to about 2.4% and/or a molar mass average value in the range from about 15,000 to about 25,000 daltons.

Guar Hydroxypropyltrimonium Chloride is a quaternary trimethyl hydroxy propyl ammonium salt based on guar gum and is added to body treatment products, especially hair treatment products, as a care substance. Due to its cationic charge Guar Hydroxypropyltrimonium Chloride absorbs on hair or skin surfaces or flocculates together with present anionic surfactants on hair or skin surfaces. In both cases this leads to a smoothing of the surface and produces an increase in gloss, easier combing, an antistatic effect and an improved skin feel.

Conventional Guar Hydroxypropyltrimonium Chloride can only be incorporated in surfactant-containing compositions in small amounts (up to about 1% by weight). At higher amounts the resulting compositions show too high viscosities and are no longer pumpable or otherwise processible.

Surprisingly, it has been discovered that Guar Hydroxypropyltrimonium Chloride, which has a nitrogen content of from about 1.9 to about 2.4% and/or a molar mass average value in the range of from about 15,000 to about 25,000 daltons, may be incorporated into a surfactant-containing base liquid even in quantities of >1% by weight without having any adverse effects on the viscosity and thus on the further processability of surfactant-containing base liquid. Guar Hydroxypropyltrimonium Chloride with a nitrogen content of from about 1.9 to about 2.4% and a molar mass average value in the range of from about 15,000 to about 25,000 daltons is available from Solvay, for example, under the name Jaguar® Optima.

The amount of Guar Hydroxypropyltrimonium Chloride, which has a nitrogen content of from about 1.9 to about 2.4% and/or a molar mass average value in the range of from about 15,000 to about 25,000 daltons, amounts preferably to between about 0.02 and about 2% by weight and more preferably to between about 0.05 and about 1.1% by weight, in each case based on the total weight of the base liquid.

In various embodiments, at least one base liquid also contains at least about 1% by weight, based on the total weight of the base liquid, of a substance with the INCI designation “Guar Hydroxypropyltrimonium Chloride”, which has a nitrogen content of from about 1.9 to about 2.4% and/or a molar mass average value in the range from about 15,000 to about 25,000 daltons.

In various embodiments, the base liquids have a viscosity of >5000 mPas (Brookfield DV 2+, spindle 5, 20 rpm, 20° C. and 60 seconds). It is preferred that the base liquids each have a viscosity in a range of from about 5,000 to about 20,000 MPas.

It is particularly preferred that the base liquids have a pseudoplastic viscosity. Structurally viscous liquids are non-Newtonian liquids whose deformation behavior can no longer be described using Newton's law. The viscosity of non-Newtonian liquids changes in particular with shear rate and/or load duration.

In various embodiments, the kit contains at least about 4, more preferably at least about 8 and most preferably at least about 10 base liquids.

The more base liquids the kit comprises, the greater the variance in the body surface treatment agents that may be prepared with them. Accordingly, individual body treatment products may be prepared that are optimally adapted to the user's needs.

In various embodiments, the base liquids each contain at least one ingredient selected from the group consisting of oils, perfumes, inorganic salts, organic acids, anti-dandruff agents, care substances, preservatives and colorants.

Surprisingly, it has been discovered that in particular high concentrations of oil, perfume and/or skin care products may be stably incorporated into a base liquid without any adverse effects, such as greatly reduced viscosity, phase separation, or the like, preventing or greatly complicating further processing, for example mixing in a mixing device.

Suitable oils include, for example, esters of linear or branched saturated or unsaturated fatty alcohols with 2-30 carbon atoms with linear or branched saturated or unsaturated fatty acids with 2-30 carbon atoms, which may be hydroxylated. These include cetyl 2-ethylhexanoate, 2-hexyl decyl stearate (for example Eutanol® G 16 S), 2-hexyldecyl laurate, isodecyl neopentanoate, isononyl isononanoate, 2-ethylhexyl palmitate (for example Cegesoft® C 24) and 2-ethylhexyl stearate (for example Cetiol® 868). Also preferred are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate.

Other preferred oils are selected from natural and synthetic hydrocarbons, especially preferably mineral oils, paraffin oils, C₁₈-C₃₀ isoparaffins.

Other preferred oils are selected from fatty alcohols with 6-30 carbon atoms which are unsaturated or branched and saturated or branched and unsaturated.

Further preferred oils are selected from the triglycerides (=triple esters of glycerol) of linear or branched, saturated or unsaturated, optionally hydroxylated C₈-C₃₀ fatty acids. The use of natural oils, e.g. amaranth seed oil, apricot kernel oil, argan oil, avocado oil, babassu oil, cotton seed oil, borage seed oil, camelina oil, thistle oil, peanut oil, pomegranate kernel oil, grapefruit seed oil, hemp oil, hazelnut oil, elderberry seed oil, currant seed oil, jojoba oil, linseed oil, macadamia nut oil, corn germ oil, almond oil, manila oil, evening primrose oil, olive oil, palm oil, palm kernel oil, Brazil nut oil, pecan nut oil, peach kernel oil, rapeseed oil, castor oil, sea buckthorn pulp oil, sea buckthorn seed oil, sesame oil, soybean oil, sunflower oil, grape seed oil, walnut oil, wild rose oil, wheat germ oil, and the liquid portions of coconut oil and the like is particularly preferred. But also synthetic triglyceride oils are preferred.

Further preferred oils are selected from the dicarboxylic acid esters of linear or branched C₂-C₁₀ alkanols, especially diisopropyl adipate, di-n-butyl adipate, di-(2-ethylhexyl)adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl)succinate.

Further preferred oils are selected from the symmetrical, asymmetrical or cyclic esters of carbonic acid with C₃-C₂₂ alkanols, C₃-C₂₂ alkanediols or C₃-C₂₂ alkanethiols, for example dicaprylyl carbonate (Cetiol® CC) or glycerol carbonate.

Further oils that are suitable are selected from the silicone oils, which include, for example, dialkyl and alkyl aryl siloxanes, such as cyclopentasiloxane, cyclohexasiloxane, dimethylpolysiloxane and methylphenylpolysiloxane, but also hexamethyldisiloxane, octamethyltrisiloxane and decamethyltetrasiloxane. Preferred silicone oils are selected from higher molecular weight linear dimethylpolysiloxanes, commercially available for example under the designation Dow Corning® 190, Dow Corning® 200 Fluid with kinematic viscosities (25° C.) in the range of from about 5-100 cSt, preferably from about 5-50 cSt or also from about 5-10 cSt, and dimethylpolysiloxane with a kinematic viscosity (25° C.) of about 350 cSt.

Perfumes used in body treatment products, in particular hair treatment products, usually comprise a complex mixture of different fragrances. Perfumes may contain, for example, aldehyde fragrances, ketone fragrances, alcohol fragrances, amine fragrances and/or ester fragrances.

The total amount of oil and perfume in a base liquid is preferably between about 0.5 and about 15% by weight and more preferably between about 3 and about 10% by weight, each based on the total weight of the base liquid.

Care substances include in particular quaternary compounds such as monomeric cationic or amphoteric ammonium compounds, monomeric amines, aminoamides, polymeric cationic ammonium compounds and polymeric amphoteric ammonium compounds.

Suitable care substances also include protein hydrolysates, vitamins, provitamins and/or vitamin precursors.

The base liquids may, in particular, further contain pH-adjusting agents. The pH value of the base liquids is preferably in the range from about 4 to about 6, more preferably in the range from about 4.5 to about 5.5.

A further subject-matter of the application is a mixing device for producing a liquid mixture, comprising a kit as contemplated herein.

The kit may be advantageously used in a mixing device for preparing a liquid mixture. The liquid mixture prepared is preferably a body treatment product, in particular a hair treatment product, preferably a shampoo, conditioner or deep conditioner.

The mixing device for preparing a liquid mixture preferably comprises a plurality of reservoirs, the reservoirs being adapted to hold a plurality of base liquids. Further, the mixing device preferably comprises at least one mixing unit, wherein the mixing unit is adapted to mix at least two base liquids from the plurality of reservoirs, and wherein the mixing unit comprises a static mixer. The mixing device preferably includes a plurality of peristaltic pumps, wherein the peristaltic pumps are adapted to convey the base liquids from the reservoirs to the mixing unit.

It may further be advantageous for the mixing device to have at least one electronic system, the electronic system being adapted to receive at least one user-specific information regarding a mixing ratio of at least two base liquids from a computer app on at least one mobile device. The mixing device is further advantageously capable of mixing the at least two base liquids according to the predefined mixing ratio by employing the plurality of peristaltic pumps and the static mixer.

### Exemplary Embodiments

TABLE 1
Composition of six basic liquids E1 to E6 as contemplated herein (in % by weight)

| Ingredient (INCI) | E1 | E2 | E3 | E4 | E5 | E6 |
|---|---|---|---|---|---|---|
| Sodium Laureth Sulfate | 8.5 | 8.5 | 12.5 | 8.5 | 8.5 | 8.5 |
| Cocamide MEA | 2.5 | 2.5 | 2.5 | 2.5 | 2.5 | 2.5 |
| Cocamidopropyl Betaine | 1.5 | 0.75 | 1 | 1.5 | 1.5 | 1.5 |
| Sodium Benzoate | 0.5 | 0.5 | 0.5 | 0.5 | 0.5 | 0.5 |
| Disodium Cocoamphoacetate | 0.4 | 0.4 | 0.25 | 0.4 | 0.4 | 0.4 |
| Citric Acid | 0.3 | 0.3 | 0.3 | 0.3 | 0.3 | 0.3 |
| Laureth-4 | 0.2 | 0.2 | 0.2 | 0.2 | 0.2 | 0.2 |
| Sodium Chloride | 0.4 | 0.4 | 0.4 | 0.4 | 0.4 | 0.4 |
| Glycol Distearate | 0.3 | 0.3 | 0.3 | 0.3 | 0.3 | 0.3 |
| Piroctone olamine | — | — | 1.6 | — | — | — |
| PEG-120 Coco Glycol | — | — | — | — | 1.2 | 1.2 |
| Glutamate | | | | | | |
| PEG-40 Hydrogenated Castor | — | — | — | — | 2.3 | 2.5 |
| Oil | | | | | | |
| Guar Hydroxypropyltrimonium | — | 0.4 | — | — | — | — |
| Chloride* | | | | | | |
| Lactic Acid | — | — | — | 1.1 | — | — |
| Magnesium chloride | — | — | — | 2 | — | — |
| DOW CORNING ® CE-8411** | — | 1.2 | — | — | — | — |
| Perfume | — | — | — | — | 2.5 | 3 |
| Colorant(s) | — | — | — | — | 0.0002 | 0.0006 |
| Water | ad 100 | ad 100 | ad 100 | ad 100 | ad 100 | ad 100 |
| pH value | 4.9 | 4.8 | 5.1 | 4.7 | 4.9 | 4.9 |
| Viscosity | 17,600 | 12,400 | 10,100 | 9,700 | 5,600 | 5,100 |

*Jaguar C-14 (ex Solvay)
**INCI name: Bis-Diisopropanolamino-PG-propyl Dimethicone/Bis-Isobutyl PEG-14 Copolymer (and) Polysorbate 20 (and) Butyloctanol

To produce a shampoo for hair which showed only a slight degree of hair damage, 60 parts of the base liquid E1, 25 parts of the base liquid E2, 5 parts of the base liquid E4 and 10 parts of the base liquid E5 were mixed in a mixing device. The shampoo obtained had a viscosity of 8,400 mPas and a pH value of 4.8.

To produce a shampoo for hair which showed a medium degree of hair damage, 5 parts of base liquid E1, 75 parts of base liquid E2, 10 parts of base liquid E4 and 10 parts of base liquid E6 were mixed in a mixing device. The shampoo obtained had a viscosity of 5,950 mPas and a pH value of 4.8.

To produce a shampoo for hair, which showed a slight degree of hair damage and dandruff, 40 parts of base liquid E1, 25 parts of base liquid E2, 25 parts of base liquid E3 and 10 parts of base liquid E5 were mixed in a mixing device. The shampoo obtained had a viscosity of 7,600 mPas and a pH value of 5.

TABLE 2
Composition of four base liquids E7 to E12 (in % by weight)

| Ingredient (INCI) | E7 | E8 | E9 | E10 | E11 | E12 |
|---|---|---|---|---|---|---|
| Sodium laureth sulfate | 8.5 | 8.5 | 12.5 | 8.5 | 8.5 | 8.5 |
| Cocamide MEA | 2.5 | 2.5 | 2.5 | 2.5 | 2.5 | 2.5 |
| Cocamidopropyl betaine | 1.5 | 1.5 | 1 | 1.5 | 1.5 | 1.5 |
| Sodium benzoate | 0.5 | 0.5 | 0.5 | 0.5 | 0.5 | 0.5 |
| Disodium coco amphoacetate | 0.4 | 0.4 | 0.25 | 0.4 | 0.4 | 0.4 |
| Citric acid | 0.3 | 0.3 | 0.3 | 0.3 | 0.3 | 0.3 |
| Laureth-4 | 0.2 | 0.2 | 0.2 | 0.2 | 0.2 | 0.2 |
| Sodium chloride | 0.4 | 0.4 | 0.4 | 0.4 | 0.4 | 0.4 |
| Glycol distearate | 0.3 | 0.3 | 0.3 | 0.3 | 0.3 | 0.3 |
| Tego Sulfosuccinate DO 75*** | — | 10 | 12 | — | — | — |
| DOW CORNING ® CE-8411** | — | — | — | — | 1.2 | 1.2 |
| Guar Hydroxypropyltrimonium | — | — | — | — | — | 1 |
| Chloride* | | | | | | |
| Guar Hydroxypropyltrimonium | — | — | — | — | 1 | — |
| Chloride**** | | | | | | |
| PEG-120 Cocoglycolglutamate | 2.0 | — | — | 2 | — | — |
| PEG-40 Hydrogenated Castor | 2.5 | 2.5 | 2.5 | 2.5 | — | — |
| Oil | | | | | | |
| Simmondsia Chinensis (Jojoba) | — | — | 1 | 1 | — | — |
| Seed oil | | | | | | |
| Perfume | 6 | 6 | 6 | 6 | — | — |
| Colorant(s) | 0.0006 | 0.0006 | 0.0006 | 0.0006 | — | — |
| Water | ad 100 | ad 100 | ad 100 | ad 100 | ad 100 | ad 100 |
| pH value | 4.9 | 4.8 | 5.1 | 4.7 | 5.0 | 4.9 |
| Viscosity | 3,800 | 7,800 | 6,200 | 1,600 | 11,300 | 29,4000 |

*Jaguar C-14 (ex Solvay)
**INCI name: Bis-Diisopropanolamino-PG-propyl Dimethicone/Bis-Isobutyl PEG-14 Copolymer (and) Polysorbate 20 (and) Butyloctanol
***INCI name: Diethylhexyl Sodium Sulfosuccinate
****Jaguar Optima (ex Solvay)

To produce a shampoo for hair which showed only a slight degree of hair damage, 60 parts of the base liquid E1, 25 parts of the base liquid E2, 5 parts of the base liquid 4 and 10 parts of the base liquid E7 were mixed in a mixing device. The shampoo obtained had a viscosity of 3,200 mPas.

For the production of a shampoo for hair, which showed only a minor degree of hair damage, 60 parts of the base liquid E1, 25 parts of the base liquid E2, 5 parts of the base liquid E4 and 10 parts of the base liquid E8 were mixed in a mixing device. The shampoo obtained had a viscosity of 8,400 mPas.

To produce a shampoo for hair which showed only a minor degree of hair damage, 60 parts of base liquid E1, 25 parts of base liquid E2, 5 parts of base liquid E4 and 10 parts of base liquid E9 were mixed in a mixing device. The shampoo obtained had a viscosity of 6,000 mPas.

To produce a shampoo for hair which showed only a slight degree of hair damage, 60 parts of the base liquid E1, 25 parts of the base liquid E2, 5 parts of the base liquid E4 and 10 parts of the base liquid E10 were mixed in a mixing device. The shampoo obtained had a viscosity of 1,300 mPas.

To produce a shampoo for hair, which showed a medium degree of hair damage, 50 parts of base liquid E1, 10 parts of base liquid E4, 10 parts of base liquid E6 and 30 parts of base liquid E11 were mixed in a mixing device. The shampoo obtained had a viscosity of 7,900 mPas and a pH value of 4.8.

To produce a shampoo for hair, which showed a medium degree of hair damage, 5 parts of base liquid E1, 10 parts of base liquid E4, 10 parts of base liquid E6 and 75 parts of base liquid E12 were mixed in a mixing device. The viscosity and pH of the shampoo obtained could not be determined.

To produce a shampoo for hair which showed a medium degree of hair damage, 20 parts of base liquid E1, 10 parts of base liquid E4, 10 parts of base liquid E6 and 60 parts of base liquid E11 were mixed in a mixing device. The shampoo obtained had a viscosity of 8,200 mPas and a pH value of 5.0.

The viscosity was determined in all cases with a Brookfield DV 2+(spindle 5, 20 rpm, 20° C. and 60 seconds).

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A kit comprising at least two base liquids for the preparation of a body treatment composition, each base liquid containing at least about 10% by weight of a surfactant mixture comprising anionic surfactants, amphoteric surfactants and nonionic surfactants.

2. The kit according to claim 1, wherein each base liquid comprises at least about 10% by weight of a surfactant mixture comprising alkyl ether sulfates, alkyl amidoalkyl betaines and alkamide monoethanolamines.

3. The kit according to claim 1, wherein the amount of alkyl ether sulfates is from about 7 to about 15% by weight, the amount of alkyl amidoalkyl betaines is from about 1.5 to about 4% by weight and the amount of alkamide monoethanolamines is from about 1 to about 2% by weight, in each case based on the total amount of base liquid.

4. The kit according to claim 1, wherein the surfactant mixture comprises sodium lauryl ether sulfate (INCI: sodium laureth sulfates), cocamidopropyl betaine (INCI: Cocamidopropyl Betaine) and N-(hydroxyethyl) coconut fatty acid amide (INCI: Cocamide Monoethanolamine).

5. The kit according to claim 1, wherein at least one base liquid further comprises a substance selected from dialkyl sulfosuccinates and dialkyl methyl sulfosuccinates.

6. The kit according to claim 5, wherein the substance is sodium dioctyl sulfosuccinate (INCI: Diethylhexyl Sodium Sulfosuccinates).

7. The kit according to claim 1, wherein at least one base liquid further comprises a substance with the INCI designation “Guar Hydroxypropyltrimonium Chloride”, which has a nitrogen content of from about 1.9 to about 2.4% and/or a molar mass average value in the range from about 15,000 to about 25,000 daltons.

8. The kit according to claim 1, wherein it comprises at least about 4 base fluids.

9. The kit according to claim 1, wherein the base liquids have a viscosity of >5000 mPas (Brookfield DV 2+, spindle 5, 20 rpm, 20° C. and 60 seconds).

10. A mixing device for producing a liquid mixture, comprising a kit according to claim 1.

11. The kit according to claim 1, wherein each base liquid contains at least about 10% by weight of a surfactant mixture consisting of anionic surfactants, amphoteric surfactants and nonionic surfactants.

12. The kit according to claim 1, wherein each base liquid contains at least about 10% by weight of a surfactant mixture consisting of alkyl ether sulfates, alkyl amidoalkyl betaines and alkamide monoethanolamines.
